(19) United States
(12) Patent Application Publication
(10) Pub. No.: US 2024/0099623 A1
(43) Pub. Date: Mar. 28, 2024

(54) SYSTEM AND METHODS FOR DIAGNOSING ATTENTION DEFICIT HYPERACTIVITY DISORDER VIA MACHINE LEARNING AND DEEP LEARNING

(71) Applicant: Eric Saewon CHANG, Seoul (KR)

(72) Inventor: Eric Saewon CHANG, Seoul (KR)

(21) Appl. No.: 17/952,272

(22) Filed: Sep. 25, 2022

(51) Int. Cl. A61B 5/16; G16H 50/70

(52) U.S. Cl. CPC A61B 5/168; G16H 50/70; G16H 50/20

(57) ABSTRACT

Various embodiments of a system and method for detecting attention deficit hyperactivity disorder (ADHD) are disclosed. According to one exemplary embodiment, a method for diagnosing ADHD may comprise processing a dataset with a natural language toolkit (NLTK) package to create preprocessed data, processing the preprocessed data with machine learning algorithm or deep learning algorithm to create processed data suitable for classification, receive patient input data from a subject patient, comparing patient input data with processed dataset to determine whether patient input data meet criteria for an ADHD classification, and diagnosing ADHD based on the comparison of the patient input data with the processed data.

### TECHNICAL FIELD

Embodiments of the present disclosure relate to a medical diagnostic system. In a particular exemplary embodiment, the present disclosure relates to a system and method for detecting a neurobehavioral disorder, such as attention deficit hyperactivity disorder (ADHD).

### DESCRIPTION OF RELATED ART

ADHD is one of the most prevalent pediatric neurodevelopmental diseases in children, and the primary features of ADHD include inattention and hyperactive-impulsive behavior. According to a national parent survey conducted in 2016, the projected number of children diagnosed with ADHD in the United States alone is approximately 6.1 million, representing about 9.4% of all children between the ages of 2 and 17. Specifically, approximately 0.4 million children between the ages of 2 and 5, approximately 2.4 million children between the ages of 6 and 11, and approximately 3.3 million children between the ages of 12 and 17 belong to this group and suffer from the ADHD symptoms.

ADHD symptoms generally appear before the age of 12, and in some children, they are noticeable as early as 3 years of age. ADHD symptoms can be mild, moderate, or severe, and they may continue into adulthood. Children with ADHD may also suffer low self-esteem, strained relationships, and poor academic achievement. Although the severity of the ADHD symptoms may reduce as they become older, some people may never fully recover from the ADHD symptoms.

To treat the ADHD symptoms, medications as well as behavioral and developmental interventions can be used. While these treatments may not fully cure ADHD, they can significantly reduce the severity of the ADHD symptoms and help patients to effectively cope with the disease to improve the quality of their lives. Further, early detection and treatment can have a significant impact on the treatment result.

Currently, however, there is no simple, straightforward method for accurately diagnosing ADHD. Physicians and specialists generally use a variety of detailed assessment methods, often involving gathering and examining of detailed information from multiple sources, conducting physical, cognitive and/or behavioral tests, and interviewing patients and their family members. Therefore, there is a need for a simple diagnostic method that can detect ADHD faster and more accurately than the currently available diagnostic methods.

There have been a number of attempts by researchers to use machine learning and deep learning to diagnose ADHD. For example, Krouska et al. (Krouska, A. et al., “Deep Learning for Twitter Sentiment Analysis: The Effect of Pre-trained Word Embedding,” Learning and Analytics in Intelligent Systems, 2020, pp. 111-124) used big data technologies to analyze vast numbers of Tweets for sentimental analysis, determining their polarity using a deep learning approach employing four well-known pre-trained word vectors: Google's Word2Vec, Stanford's Crawl GloVe, Stanford's Twitter GloVe, and Facebook's FastText. According to their study, deep learning outperformed typical machine learning algorithms for Tweets classification. Therefore, deep learning models were applied to three different famous Tweets-related datasets. The STS-Gold dataset was made up of random Tweets with no particular topic focus, whereas the OMD and HCR datasets included Tweets from specified topics. In terms of pre-trained word embeddings, FastText generated more consistent results across datasets, which was 83.65%, although Twitter GloVe obtained very high accuracy rates despite its lower dimensionality.

Ahmad et al. (Ahmad, H. et al., “Applying Deep Learning Technique for Depression Classification in Social Media Text,” Journal of Medical Imaging and Health Informatics, 10(10), 2020, pp. 2446-2451) employed deep learning models to detect depression with a tweet dataset. They ran trials with several machine learning and deep learning models and assessed their performance using a public dataset. Their primary objective is to detect depression using a deep learning methodology based on the BiLSTM method. They used the textual content obtained from Twitter as a benchmark dataset. Since the label comprises either normal or depressed, this study falls under binary categorization. The results are promising, demonstrating that the BiLSTM outperformed other approaches in terms of f-measure (90%), recall (91%), accuracy (93%), and precision (89%).

Hamdi et al. (Hamdi, E. et al. “A Convolutional Neural Network Model for Emotion Detection from Tweets,” Advances in Intelligent Systems and Computing, 2018, pp. 337-346) investigated emotion recognition in Tweets. They employed a convolutional neural network to classify the labels (CNN). The system was tested by categorizing sentiment into positive and negative categories using the Stanford Twitter Sentiment dataset, collected via Twitter Search API. For training, 80K randomly chosen sentences are gathered, with additional 16K sentences collected for validation and positive labels outnumber negative ones by a factor of two. The maximum sentence length has been reduced to 8, while the vocabulary size has been increased to 50,485. The accuracy of the presented model was 80.6%. CNN has been shown to produce excellent results without the requirement for an additional dataset or an extra model to create word vectors.

Neethu et al. (Neethu, M. S. et al., “Sentiment Analysis in Twitter Using Machine Learning Techniques,” 2013 Fourth International Conference on Computing, Communications and Networking Technologies, ICCCNT) investigated sentiment analysis on Twitter using machine learning methods. According to their findings, the prevalence of slang phrases and misspellings makes analyzing Twitter sentiment more challenging than conventional sentiment analysis. The authors proposed two noel solutions to alleviate these drawbacks. The first step is to extract and integrate Twitter-specific features into the feature vector. Following that, these features are eliminated from tweets, and extracted features are performed again as if it were on regular text. For classification, they used the SVM Classifier, Naive Bayes, Maximum Entropy, and Ensemble algorithms, which returned 89.5%, 90%, 90%, and 90%, respectively. This study identifies the impact of domain information on sentiment analysis.

### SUMMARY

Unfortunately, however, all of the above-discussed methods have had various shortcomings, especially in the practical application in patients. Accordingly, various exemplary embodiments of the present disclosure provide an improved system and method for diagnosing ADHD using improved machine learning and deep learning approaches. Machine learning generally refers to a data analysis method implemented in a computer system as algorithms that allow the computer system to parse data, learn from the data, identify certain patterns in the data, and apply information learned from the data to make decisions without substantial intervention from human. Deep learning generally refers to a type of machine learning that structures algorithms in layers that can be used to progressively extract higher-level features from the data.

To attain the advantages and in accordance with the purpose of the invention, as embodied and broadly described herein, one aspect of the invention may provide a method for diagnosing ADHD. The method may comprise processing a dataset with a natural language toolkit (NLTK) package to create preprocessed data, processing the preprocessed data with machine learning algorithm or deep learning algorithm to create processed data suitable for classification, receive patient input data from a subject patient, comparing patient input data with processed dataset to determine whether patient input data meet criteria for an ADHD classification, and diagnosing ADHD based on the comparison of the patient input data with the processed data.

Additional objects and advantages will be set forth in part in the description which follows, and in part will be obvious from the description, or may be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF DRAWINGS

The accompanying drawings, which are incorporated in and constitute a part of this specification, illustrate several embodiments of the invention and together with the description, serve to explain the principles of the invention.

FIG. 1 is an operational flow chart illustrating an exemplary diagnostic system and method for diagnosing ADHD, according to one exemplary embodiment.

FIG. 2 is an overall description of an exemplary dataset from the Kaggle website.

FIG. 3 is a result of a kernel distribution estimation plot from the ‘score’ column in the dataset.

FIG. 4 is the result of word cloud from the ‘selftext’ column in the dataset.

FIG. 5 is an exemplary overall design of an extra tree algorithm, according to one exemplary embodiment.

FIG. 6 is a flow chart illustrating an exemplary data processing, according to some exemplary embodiments.

FIG. 7 is a comparison of various machine learning models based on accuracy scores when “selftext” is used as the features.

FIG. 8 is a comparison of various machine learning models based on accuracy scores when “title” is used as the features.

### DESCRIPTION OF EXEMPLARY EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

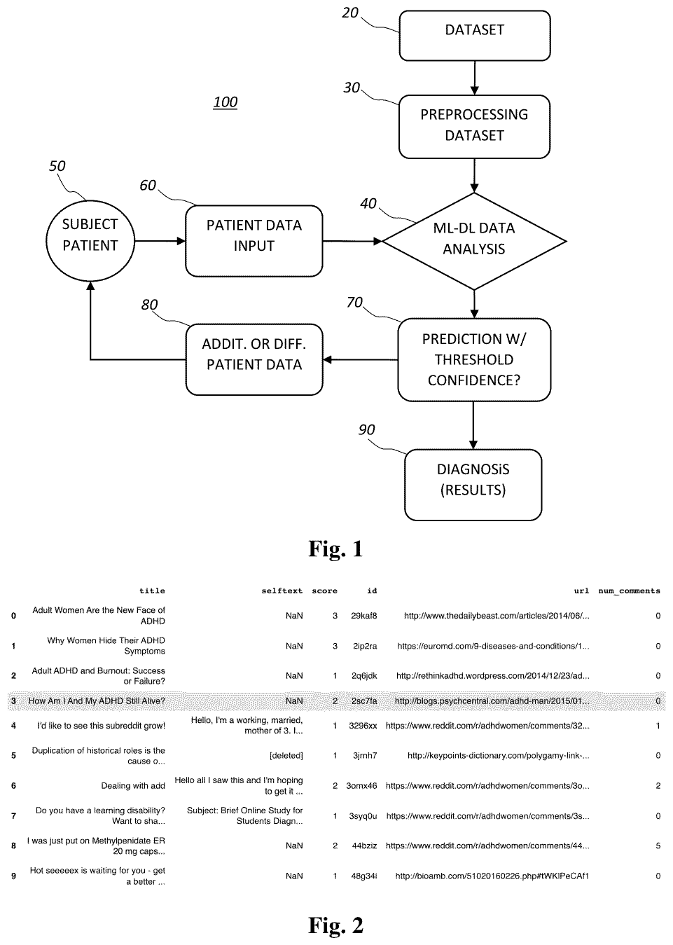

FIG. 1 is an operational flow chart schematically illustrating an exemplary diagnostic system 100 and related method for diagnosing ADHD using machine learning and deep learning approach according to one exemplary aspect of the present disclosure. Although the present disclosure is described in connection with diagnosing ADHD, it should be understood that the diagnosing system and method consistent with the present disclosure may be used to diagnose other suitable neurodevelopmental disorders.

As shown in FIG. 1, an exemplary diagnostic system 100 may be configured to diagnose ADHD of a subject patient 50 by analyzing input data from subject patient 50, comparing the analyzed data with the dataset preprocessed by a machine learning and deep learning method, and generating a predicted classification of the subject patient with a threshold confidence level. Machine learning and deep learning enables exploiting large datasets to generate predictive models by developing a target outcome based on a set of predictors or features in existing data.

System module 100 may comprise a preprocessing module 30 configured to preprocess a dataset 20. According to one exemplary embodiment, dataset 20 may comprise data gathered from published community datasets available from one or more online community data platform, such as, Kaggle. For example, the community datasets may comprise all Reddit posts and comments from one or more subreddits discussing ADHD. Dataset 30 may be in the form of CSV files. In some cases, rows with NaN values in the CSV files, which are generated when arithmetic operations result in undefined or unrepresentable values, may be removed for faster processing.

Alternatively or additionally, dataset 20 may comprise real-world clinical data that are collected from available medical records of individuals with and without ADHD. In some exemplary embodiments, dataset 20 may be prepared by directly collecting data from groups of participants with and without ADHD. For example, one set of data may be collected from a group of individuals pre-diagnosed with and met the criteria for ADHD. Another set of data (e.g., control data) may be collected randomly from a group of individuals with no ADHD or any other known neurological disorders.

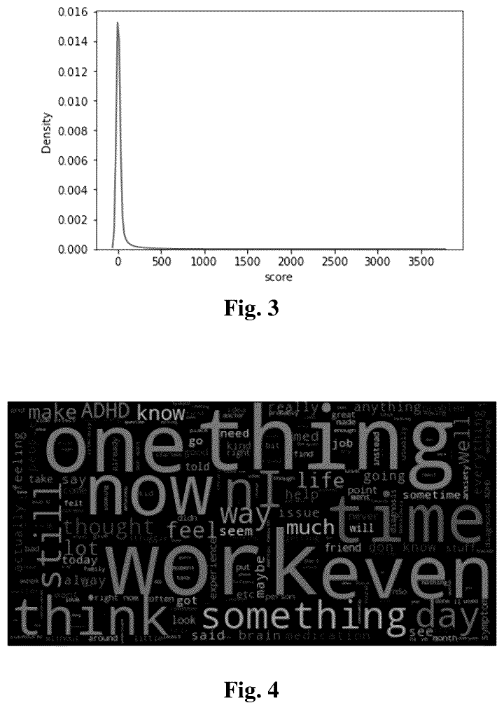

In the exemplary embodiment that used the Kaggle data as dataset 20, dataset 20 is preprocessed to generate two columns of data—i.e., “selftext” and “score,” as shown in FIG. 2. FIG. 3 shows a kernel distribution estimation (KDE) plot, which depicts the probability density function of the continuous or non-parametric data variables, from the “score” column in dataset 20. Because the “score” column encompasses a wide range of values, as indicated in the KED plot shown in FIG. 3, creating the precise classification of the labels may not be practically feasible. Instead, simple selection of labels ranging from 1 to N (e.g., N=5) can be used. FIG. 4, which is a word cloud from the “selftext” column, shows the most frequently used word in the specific column based on Reddit ADHD dataset.

FIG. 5 schematically illustrates an overall design of an exemplary extra tree algorithm, according to another aspect of the present disclosure. The extra tree is a decision tree-based ensemble algorithm, which may function similar to the random forest algorithm in machine learning. One of the main differences between an extra tree algorithm and a random forest algorithm is whether the algorithms utilize a portion of the dataset at a time or the entire dataset at once. For example, a random forest algorithm utilizes a bagging method, which is an abbreviation of bootstrap aggregating as it takes samples several times from the entire dataset and learns each model to aggregate the results via majority voting. On the other hand, an extra tree algorithm utilizes the entire dataset at once. Another difference is the use of cut-points for splitting nodes or classification. A random forest algorithm selects the best split points for splitting nodes, whereas an extra tree algorithm randomly selects split points for splitting nodes. As the extra tree algorithm does not attempt to locate the most efficient split for the classification, it can reduce the speed of the algorithm even though it utilizes the whole dataset.

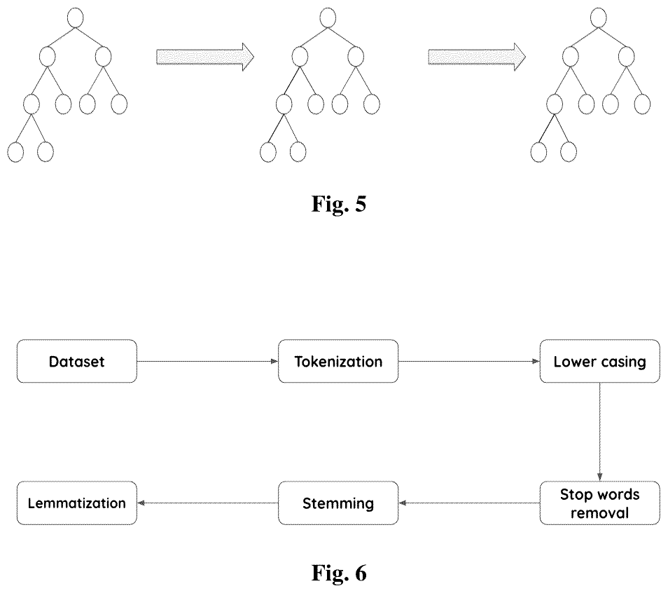

Before dataset 20 is analyzed through machine learning and deep learning models in a data analysis module 40 (see FIG. 1), additional preprocessing procedures may be carried out in order to obtain more efficient and reliable results. For example, FIG. 6 illustrates an exemplary flow chart for additional data processing according to one exemplary embodiment of the present disclosure. As shown in the figure, the additional preprocessing may comprise tokenization utilizing the Natural Language Toolkit (NLTK) package. The preprocessing may also comprise converting all characters in dataset 50 to lower case and deleting stop words in English. The preprocessing may also comprise stemming to produce morphological variants of a root or base word and lemmatizing to group together different inflected forms of a word. Stemming and lemmatizing can be performed by utilizing the NLTK's built-in PerterStemmer and WordNetLemmatizer functions.

After preprocessing dataset 20 in preprocessing module 30, dataset 20 is analyzed through one or more machine learning and deep learning models already known and available in the art. FIG. 7 shows the comparison of various machine learning models based on accuracy score (feature: “selftext”) from an exemplary dataset 20 gathered from the Kaggle platform. As shown in the figure, ExtraTreesClassifier yielded the highest accuracy rate of 81.49%, followed by RandomForestClassifier of 80.33%, LGBMCIassifier of 75.77%, SVC of 74.06%, DecisionTreeClassifier 58.72%, KNeighborsClassifier 54.43%, Logistic Regression 53.21%, and GaussianNB 20.78%. ExtraTreesClassifier and RandomForestClassifier are both ensemble machine learning algorithms based on a decision tree. In general, the ExtraTreesClassifier produces substantially quicker results.

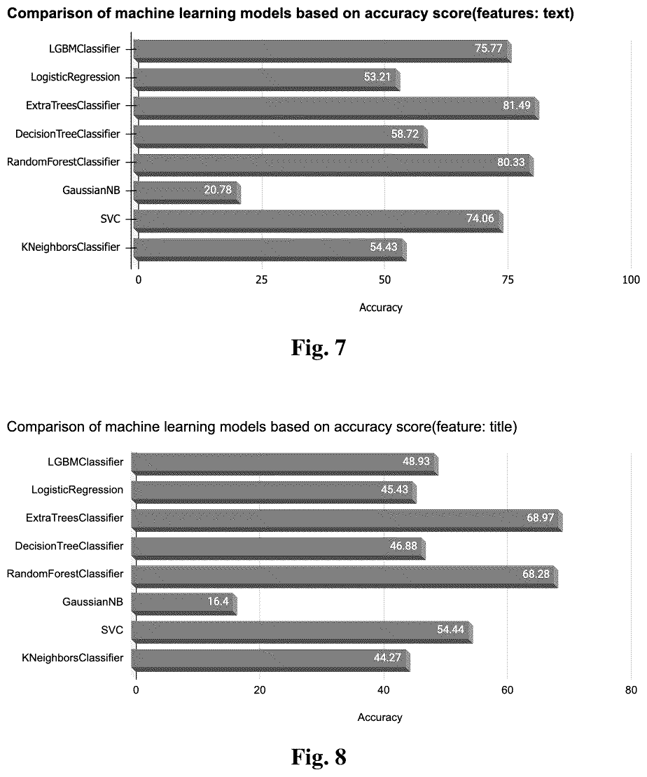

In some exemplary embodiments, the title column (see FIG. 2) can be included as an input variable. When the title column was included as an input variable, the models' accuracy score may drop considerably. For example, as shown in FIG. 8, ExtraTreesClassifier had the highest accuracy rate of 68.97%, followed by RandomForestClassifier with 68.28%, SVC with 54.44%, LGBMCIassifier with 48.93%, DecisionTreeClassifier with 46.88%, Logistic Regression with 45.43%, KNeighborsClassifier with 44.27%, and GaussianNB with 16.4%.

Referring back to FIG. 1, once dataset 20 is analyzed through machine learning and deep learning models with ADHD classifications or features, patient data is input into data analysis module 40 via a suitable patient data input module 60. Patient data input module 60 may be a traditional input device, such as a keyboard, or a data transmission device, such as a USB connecting device or storage device. The patient data may comprise various feature values input by subject patient 50. For example, the patient data may comprise sampling of tweets or other social networking postings by subject patient 50. The patient data may also comprise answers provided by subject patient 50 obtained in response to a series of targeted questionnaires. Alternatively or additionally, the patient data may comprise speech data of subject patient 50 in a conversation in normal day settings.

The patient data are then input into data analysis module 40 to compare with the analyzed result of dataset 20 with a predetermined set of ADHD classifications. A prediction module 70 determines whether the patient data meets criteria for ADHD classification above a predetermined threshold confidence level. In one exemplary embodiment, the threshold confidence level may be set to be above 70%. If the prediction accuracy is above the threshold confidence, prediction module 70 may transmits the result of the diagnosis through a diagnosis output module 90, such as, for example, a wired or wireless display terminal or printer.

On the other than, if the prediction accuracy is below the threshold confidence level, system 100 may request an additional and/or different type of patient data from subject patient 50 via a request module 80, such as, for example, a wired or wireless display screen, cell phone, tablet, signal terminal, or printer. Subject patient 50 may then supply additional patient data and the process repeats until a prediction meeting the threshold confidence is obtained.

The system and method according to the present disclosure enable machine learning algorithms, such as the Extra Tree algorithm, to diagnose ADHD disease and even classify the level of ADHD. For example, the Extra Tree algorithm achieved an 81% of accuracy score with the “selftext” column, which contains the tweets data. On the other hand, when only the “title” column was used, the accuracy score got decreased to 68.97%. Furthermore, as the machine learning models were utilized as classifiers, this could reduce time compared to deep learning classifiers. These advantages could allow doctors and therapists to diagnose the ADHD disease even with simple tweets and this method would be more efficient compared to the conventional diagnosis methods. The present disclose also provides a faster and more accurate ADHD diagnosis procedure than the existing ADHD test.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for diagnosing attention deficit hyperactivity disorder (ADHD) comprising:
processing a dataset with a natural language toolkit (NLTK) package to create preprocessed data;
processing the preprocessed data with machine learning algorithm or deep learning algorithm to create processed data suitable for classification;
receive patient input data from a subject patient;
comparing patient input data with processed dataset to determine whether patient input data meet criteria for an ADHD classification; and
diagnosing ADHD based on the comparison of the patient input data with the processed data.

2. The method of claim 1, wherein diagnosing ADHD comprises classifying the level of ADHD.

3. The method of claim 1, wherein the dataset comprises data collected from one or more social networking sites.

4. The method of claim 1, wherein the preprocessing comprises at least one of tokenization, lower casing, deleting stop words, stemming, and lemmatization.

5. The method of claim 1, wherein the machine learning algorithm comprises Extra Tree.

6. The method of claim 1, further comprising determining a confidence level of the diagnosis.

7. The method of claim 6, wherein determining the confidence level of the diagnosis comprises determined whether the confidence level of the diagnosis is above a predetermined threshold confidence level.

8. The method of claim 7, wherein the predetermined threshold confidence level is above 70%.
